(19) United States
(12) Patent Application Publication
(10) Pub. No.: US 2026/0035323 A1
(43) Pub. Date: Feb. 5, 2026

(54) PROCESS OF PRODUCING TOLUENE

(71) Applicant: UOP LLC, Rosemont, IL (US)

(72) Inventor: David Lafyatis, Schaumburg, IL (US)

(21) Appl. No.: 19/231,147

(22) Filed: Jun. 6, 2025

Related U.S. Application Data
(60) Provisional application No. 63/677,944, filed on Jul. 31, 2024.

(51) Int. Cl. C07C 5/41

(52) U.S. Cl. CPC C07C 5/417

(57) ABSTRACT

A process of producing toluene is disclosed. The process comprises contacting a hydrocarbonaceous feed stream with a dehydrogenation catalyst to produce a dehydrogenated effluent stream. The catalyst has been prepared to have a low surface area from about 90 to 140 m2/g. A reduced amount of heavier hydrocarbons are produced when this catalyst is used. The dehydrogenated effluent stream is separated into a vapor stream comprising hydrogen and a liquid stream. The liquid stream is fractionated to provide a fractionator overhead stream comprising C7-hydrocarbons and a fractionator bottoms stream comprising toluene. The fractionator overhead stream is passed to a stabilizer column to provide an offgas stream.

### FIELD

The field is the process of producing toluene. The field may particularly relate to a process of reducing the formation of heavier hydrocarbons in a dehydrogenation process.

### BACKGROUND

Hydrogen is expected to have significant growth potential because it is a clean-burning fuel. However, hydrogen production processes based on steam reforming, autothermal reforming, partial oxidation, or gasification of hydrocarbon or carbonaceous feedstocks are significant emitters of carbon dioxide. Government regulations and societal pressures are increasingly taxing or penalizing carbon dioxide emissions or incentivizing carbon dioxide capture. Hydrogen produced with energy from solar and wind and from electrolysis of water does not involve the production of carbon dioxide, could meet projected global energy demand in the future and play a vital role in reducing carbon dioxide emissions. The recently renewed interest in alternative energy sources and energy carriers opens up new prospects for this process to be applied as a feed system for fuel cells, power generation and many more applications.

Hydrogen is a clean, efficient energy carrier in various mobile fuel-cell applications and has no adverse effects on the environment and human health. With increase in global demand for hydrogen, solutions need to be developed to transport hydrogen especially to locations which are renewable depleted. Hydrogen generated by renewable energy sources is called green hydrogen. Green hydrogen is expected to be an important element in the future carbon-neutral economy and may need to be transported to locations as far as 8000 km from the source of generation.

There exists a huge regional disparity in the cost of production of hydrogen. A number of technologies have been developed for transporting hydrogen, including ammonia, liquid hydrogen, and liquid organic hydrogen carrier (LOHC) to address this disparity. Toluene-methylcyclohexane (MCH) is expected to be a significant player in LOHC considering numerous advantages, such as easy integration into the existing fuel sector supply chain and distribution network, utilization in idle refinery assets, flexibility for co-processing, and higher relative safety handling.

LOHC involves the reversible dehydrogenation reaction of methylcyclohexane (MCH) to produce toluene (TOL) and hydrogen. It has been proposed as a solution for the storage, transportation, and distribution of hydrogen produced from renewable energy sources. For power generation, the hydrogen from this process is usually compressed for a downstream power generation unit. Usually, purity requirements for a power generation unit is very stringent. Due to the relatively high cost associated with the green hydrogen production, it is necessary to recover almost all hydrogen.

The process of converting MCH to toluene and hydrogenation is highly selective but may lead to the formation of byproducts that will build up in the carrier loop. These byproducts need to be removed by distillation before they cause problems with catalyst or equipment life. The energy required to remove these byproducts must be minimized to avoid consuming valuable hydrogen product or consuming energy from carbon-emitting sources.

Accordingly, there is a need to have more effective and efficient ways to purify and transport hydrogen and in particular hydrogen produced from a renewable resource. It has now been found that the use of a catalyst having alkaline earth or alkali metals and a reduced catalyst surface area can significantly reduce the production of undesirable byproducts such as heavier hydrocarbons.

### BRIEF SUMMARY

A process for producing toluene is provided. The process comprises contacting a hydrocarbonaceous feed stream with a dehydrogenation catalyst to produce a dehydrogenated effluent stream. The gamma alumina used for the dehydrogenation catalyst has been calcined sufficiently at an elevated temperature to reduce the catalyst surface area to less than 140 m²/gm. The catalyst surface area is preferably from 90 to 140 m²/gm and preferably from about 120 to 140 m²/gm. Then the gamma alumina support is impregnated with platinum and an alkaline earth or alkaline metal followed by oxidation and then reduction before being used in the dehydrogenation reactor. The dehydrogenated effluent stream is separated into a vapor stream comprising hydrogen and a liquid stream. The dehydrogenated effluent liquid stream contains less than about 100 ppm more than the hydrocarbonaceous feed stream and preferably less than about 80 ppm more than the hydrocarbonaceous feed stream of hydrocarbons that are heavier than C₁₀. In general, the rate of production of these heavier hydrocarbons is reduced by at least 50% when compared to the use of catalysts having a higher surface area. The liquid stream is fractionated to provide a fractionator overhead stream comprising C7-hydrocarbons and a fractionator bottoms stream comprising toluene. The fractionator overhead stream is passed to a stabilizer column to provide an offgas stream.

### BRIEF DESCRIPTION OF THE DRAWINGS

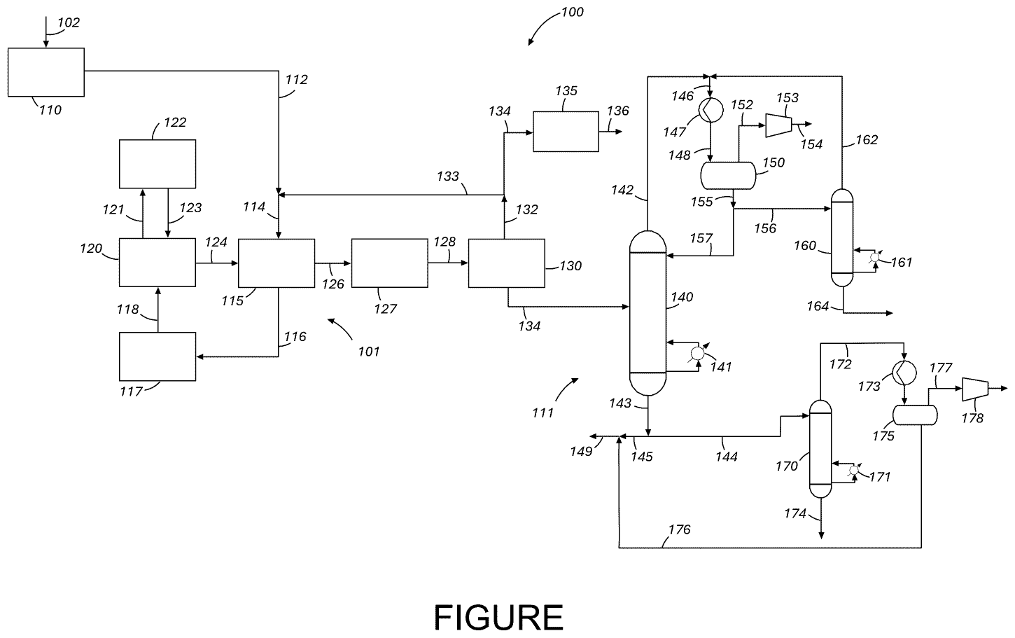

The FIG. 1s a schematic drawing of an exemplary embodiment of a process and apparatus of the present disclosure.

### Definitions

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” or “directly” means that fluid flow from the upstream component enters the downstream component without passing through any other intervening vessel.

The term “indirect communication” means that fluid flow from the upstream component enters the downstream component after passing through an intervening vessel.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

The term “Cx” is to be understood to refer to molecules having the number of carbon atoms represented the subscript “x”. Similarly, the term “Cx−” refers to molecules that contain less than or equal to x and preferably x and less carbon atoms. The term “Cx+” refers to molecules with more than or equal to x and preferably x and more carbon atoms.

### DETAILED DESCRIPTION

The liquid organic hydrogen carrier (LOHC) process involves hydrogenating toluene to methylcyclohexane (MCH) at a first location, transferring the MCH to a second location, and dehydrogenating the MCH to toluene and hydrogen at the second location. To ensure minimal loss of hydrogen and overall process efficiency, the dehydrogenation process must be performed with no by-products or efficiently removing the by-products. MCH acts as a liquid organic hydrogen carrier, and it can be transferred in storage vessels and/or pipelines for several thousands of miles to the final destination with very minimal to no degradation. The LOHC process helps address the supply and demand gap of blue and green hydrogen as well as the huge differential cost of production between regions.

The present disclosure provides a dehydrogenation catalyst, process of making the catalyst and a process that results in a reduced amount of heavier hydrocarbons being produced in the dehydrogenation process.

A hydrocarbon stream comprising the methylcyclohexane may be produced at a location and transferred to a second location. The methylcyclohexane feed from the storage tanks which may not be completely dry or nitrogen-blanketed may be treated in an oxygen stripper before being routed to the dehydrogenation reactor section. The methylcyclohexane feed may be mixed with a recycle methylcyclohexane stream for example which may be taken from a purification section. The methylcyclohexane feed may be mixed with the hydrogen and preheated for example by exchange with a reactor effluent stream. The temperature of the methylcyclohexane feed may be raised to the reaction temperature in the convection section of a charge heater and passed to the dehydrogenation reactor section. The dehydrogenation reactor section may comprise one or more dehydrogenation reactor(s). The methylcyclohexane feed is charged to the dehydrogenation reactor(s). The dehydrogenation reactor(s) may be radial flow reactors. Interheaters may be used to raise the reactor effluent back to the desired reactor inlet temperature for the next dehydrogenation reactor. The effluent from the last dehydrogenation reactor is cooled in a combined feed exchanger and the product condenser before passing to the separator.

In the separator, the reactor effluent is separated into vapor and liquid streams. The separator vapor may be separated into net gas and recycle gas. The recycle gas may be sent back to be mixed with the feed. The net gas is the hydrogen gas product stream which may be passed to the hydrogen gas compression section. Toluene-rich liquid from the separator is pumped to the purification section.

The hydrogen gas compression section may comprise one or more hydrogen gas compressor(s) which provide sufficient pressure to meet hydrogen purity requirements. The hydrogen purity increases through each stage of compression.

The purification section may comprise a deheptanizer column and a stabilizer column. The present disclosure provides passing the toluene-rich liquid from the separator first to the deheptanizer column. Applicants found that placing the deheptanizer column upstream of the stabilizer significantly reduces the reflux stream to both the deheptanizer column and the stabilizer column. Also, the disclosed process reduces the number of stages required in the deheptanizer column for the separation of toluene. Also, the deheptanizer column is operated at a pressure as low as possible order to minimize reboiler duty and the column temperature. Moreover, the purification section of the present disclosure comprises a rerun column to remove heavy contaminants. A vent compressor is required to recover the small vapor stream from the deheptanizer column and deliver it to the fuel gas system. The rerun column may also be operated at a pressure as low as possible to be able to recover as much toluene as possible.

In the deheptanizer column, toluene is separated in the toluene-rich liquid bottoms stream. The overhead stream comprising C7-hydrocarbons and light gases is condensed and passed to the stabilizer column. A reflux stream is taken from the condensed liquid stream. The stabilizer column separates offgas comprising hydrogen, methane and traces of C5-6 hydrocarbons from a liquid stream comprising C6-7 hydrocarbons, dimethylcyclopentane (DMCP), and ethylcyclopentane (ECP). The offgas stream can be sent to the refinery or used as fuel gas, for example.

The toluene-rich liquid bottoms stream may be passed to the rerun column to separate a heavy stream comprising C8-16 hydrocarbons and provide a toluene product stream.

The FIGURE illustrates an exemplary embodiment of the process of producing toluene 100. The process 100 comprises a dehydrogenation reaction section 101 and a purification section 111. A hydrocarbonaceous stream is taken from a storage tank in line 102 and passed to the dehydrogenation reaction section 101. In an aspect, the hydrocarbonaceous stream in line 102 may be taken from a saturation unit. The feed stream to the saturation unit may comprise an aromatic hydrocarbons stream and a hydrogen stream produced from renewable sources for example “green hydrogen” or “blue hydrogen”. In an exemplary embodiment, the hydrocarbonaceous stream in line 102 comprises MCH. In another exemplary embodiment, the hydrocarbonaceous stream in line 102 comprises one or both of MCH and toluene. In an aspect, the dehydrogenation reaction section 101 may comprise an oxygen stripper 110, a combined feed exchanger 115, a charge heater 117, and a dehydrogenation reactor 120.

The hydrocarbonaceous stream in line 102 is passed to the oxygen stripper 110 to remove oxygen and provide a stripped hydrocarbonaceous stream in line 112. The stripped hydrocarbonaceous stream in line 112 may be combined with a recycle hydrogen stream in line 133 forming a combined feed stream in line 114. The combined feed stream in line 114 may be passed to a combined feed exchanger 115 to preheat the combined feed stream in line 114 by heat exchange with a dehydrogenation reactor effluent stream in line 124. In an embodiment, the oxygen stripper 110 is optional and the hydrocarbonaceous stream in line 102 may be combined with the recycle hydrogen stream to provide the combined feed stream in line 114. A preheated combined feed stream is taken in line 116 from the combined feed exchanger 115. The preheated combined feed stream in line 116 may be further heated in a charge heater 117 to the reactor temperature. A heated combined feed stream is taken in line 118 from the charge heater 117 and passed to the dehydrogenation reactor 120. The dehydrogenation reactor 120 may comprise one or more dehydrogenation catalyst bed(s). In an aspect, the dehydrogenation reactor 120 may comprise one or more dehydrogenation reactor vessels to dehydrogenate the heated combined feed stream. In another aspect, the dehydrogenation reaction section 101 may comprise one or more interheater(s) 122 to heat an intermediate reactor effluent stream in line 121 to the desired reactor inlet temperature between dehydrogenation reactors. A heated intermediate reactor effluent stream may be taken in line 123 from the interheater 122 and send back to the dehydrogenation reactor 120.

The hydrocarbonaceous feed 102 typically comprises methylcyclohexane (MCH). This stream may be produced from a number of different processes. In the exemplary embodiment, it is envisioned that the MCH may be produced in a toluene saturation unit that will chemically bind green or blue hydrogen to a toluene stream. It is further envisioned that this toluene stream may be chemically similar to a toluene product stream of the process as described later in detail or be the toluene product stream of the process itself.

Any suitable dehydrogenation catalyst that can achieve a selectivity in the dehydrogenation of methylcyclohexane to toluene and hydrogen in excess of 99 can be used in the dehydrogenation reactor 120. Suitable dehydrogenation catalysts may include, but are not limited to, alumina, a noble metal, and an alkali or alkaline earth metal. Suitable noble metals include, but are not limited to, platinum, palladium, rhodium, ruthenium, rhenium, Iridium, gold, osmium, silver, or combinations thereof. Suitable alkali or alkaline earth metals include, but are not limited to, sodium, cesium, potassium, rubidium, francium, lithium, beryllium, strontium, barium, calcium, magnesium, radium, or combinations thereof. The catalyst most often has a support material such as gamma alumina, which is in a spherical or extrudate form. The gamma alumina is calcined at an elevated temperature of about 650° C. to 815° C. The gamma alumina that is produced has a surface area of less than about 140 m²/g. Preferably the surface area is from 90-140 m²/g and most preferably from about 120-140 m²/g. In one embodiment, the catalyst support is impregnated with Pt at about 0.1-2.0 wt % and K or Li at about 0.2-2.5 wt %. preferably the catalyst comprises 0.2-4.0 wt % alkaline earth metal or alkaline metal. Preferably the catalyst comprises potassium, lithium or a combination thereof. The impregnation can be done in a single step or in several sequential steps. After impregnation, the catalyst is oxidized and reduced before being used in the dehydrogenation reactor to produce the dehydrogenation product (toluene) and hydrogen. A lower amount of heavier hydrocarbon materials are produced with less than 150 ppm hydrocarbons greater than C10 produced in the reactor section. Preferably less than 100 wt ppm hydrocarbons greater than C10 are produced and most preferably less than 50 wt ppm hydrocarbons greater than C10 are produced. The lower the amount of the heavier hydrocarbons that are produced, the better.

A dehydrogenated reactor effluent stream is taken in line 124 from the dehydrogenation reactor 120 and passed to the combined feed exchanger 115. A heat exchanged perhaps cooled dehydrogenated reactor effluent stream is taken in line 126 from the combined feed exchanger 115. The heat exchanged dehydrogenated reactor effluent stream in line 126 may be passed to a cooler 127 to provide a cooled dehydrogenated reactor effluent stream in line 128. The reactions taking place in the dehydrogenation reactor 120 may include a main reaction of dehydrogenation of the MCH to toluene releasing hydrogen and may include one or more side reactions. One side reaction includes cracking of toluene to benzene and methane. Another side reaction includes isomerization of methylcyclohexane to dimethylcyclopentane (DMCP), and ethylcyclopentane (ECP). A third side reaction includes the dimerization of methylcyclohexanes to heavier molecules, such as dimethyl biphenyl compounds. These side reactions affect the overall yield and the purity of the products streams of the process 100. The purification section 111 separates byproducts from the products streams to promote the purity of the product streams while consuming a minimum energy in the separation.

The dehydrogenation reaction section 101 further comprises a separator 130 to separate the reactor effluent into vapor and liquid streams. The cooled dehydrogenated reactor effluent stream in line 128 is passed to the separator 130 where it is separated into a separator vapor stream comprising hydrogen in line 132 and a separator liquid stream comprising toluene in line 134. The recycle hydrogen stream is taken in line 133 from the separator vapor stream. The remaining portion of the separator vapor stream is taken in line 134 and passed to a hydrogen compressor 135 where it is compressed to provide a hydrogen product stream which is taken in line 136.

The separator liquid stream in line 134 is a toluene rich liquid stream, and toluene is separated from this stream. The separator liquid stream in line 134 is passed to the purification section 111.

In an embodiment, the purification section 111 comprises a fractionation column 140, a stabilizer column 160, and a rerun column 170.

In an embodiment, separator liquid stream in line 134 is fractionated in the fractionation column 140 to provide a fractionator overhead stream in line 142 and a fractionator bottoms stream in line 143. In an exemplary embodiment, the fractionation column 140 is a deheptanizer column. The fractionator overhead stream in line 142 may comprise C7-hydrocarbons. In an embodiment, the fractionator overhead stream in line 142 may comprise benzene and C7-saturated hydrocarbons including hexanes, methylcyclopentane, cyclohexane, heptane, dimethylcyclopentane, methylcyclohexane, and ethylcyclopentane. In another embodiment, the fractionator overhead stream in line 142 may comprise light hydrocarbons for example C3-hydrocarbons, and/or C2-hydrocarbons. Some toluene may slip to the fractionator overhead stream in line 142. The fractionator overhead stream in line 142 may also comprise trace amounts of hydrogen and methane.

In an exemplary embodiment, the fractionation column 140 may be operated at an overhead pressure of about 34 kPa (g) (5 psig) to about 207 kPa (g) (30 psig) and a bottoms temperature of about 93° C. (200° F.) to about 177° C. (350° F.). In an aspect, the heat to the fractionation column 140 may be supplied from a reboiler 141 using steam. Operating the fractionation column 140 at a lower pressure minimizes the duty of the reboiler 141. A side draw may be removed from fractionation column 140 to remove impurities or used in the production of gasoline.

The fractionator overhead stream in line 142 may be passed to the stabilizer column. In an aspect, the fractionator overhead stream in line 142 may be combined with a stabilizer column overhead stream in line 162 to provide a combined overhead stream in line 146. The combined overhead stream in line 146 may be cooled in an overhead heat exchanger 147 and a cooled overhead stream in line 148 is passed to an overhead receiver 150. The cooled overhead stream in line 148 may be totally or partially condensed in the overhead receiver 150 to provide a condensed overhead liquid stream in line 155 and an off gas stream in line 152. A reflux stream is taken in line 157 from the condensed overhead liquid stream in line 155. In an embodiment, the reflux stream in line 157 may be passed to the fractionation column 140 at a reflux to feed ratio of about 0.05 to 0.5. In another embodiment, the reflux stream in line 157 may be passed to the fractionation column 140 at a reflux to feed ratio of about 0.045 to 0.4. In another embodiment, the reflux stream in line 157 may be passed to the fractionation column 140 at a reflux to feed ratio of about 0.04 to 0.2 or about 0.02 to 0.2.

The present process does not require a recycle stream of methylcyclohexane from the fractionation column 140 for recycle to the dehydrogenation reactor 120 to ensure complete conversion of the methylcyclohexane to toluene and hydrogen. With no recycle stream from the fractionation column 140, the fractionation column of the current process may be run at a much lower reflux to feed ratio. Also, the fractionation column 140 of the current process requires a much smaller number of stages even as low as 50% of the number of stages when compared with a fractionation column with a recycle stream.

A fractionator overhead liquid stream is taken in line 156 from the condensed overhead liquid stream in line 155. The fractionator overhead liquid stream in line 156 is a net liquid stream from the overhead receiver 150. The net liquid stream in line 156 is passed to the stabilizer column 160. The stabilizer column removes the byproducts and/or impurities from the process in a bottoms stream in line 164. In an aspect, the stabilizer column bottoms stream in line 164 may comprise benzene and C7-saturated hydrocarbons such as hexane, methylcyclopentane, cyclohexane, heptane, dimethylcyclopentane, methylcyclohexane, and ethylcyclopentane. The stabilizer column overhead stream in line 162 may comprise hydrogen and methane. The stabilizer column overhead stream in line 162 may be recycled. The stabilizer column overhead stream in line 162 may be combined with the fractionator overhead stream in line 142 to provide the combined overhead stream in line 146 and passed to the overhead receiver 150. The offgas stream may be taken in line 152 from the overhead receiver 150, compressed in an overhead compressor 153 to provide a compressed offgas stream in line 154. The compressed offgas stream in line 154 may comprise hydrogen and methane. The compressed offgas stream in line 154 may be used in the process, for example, burned to provide heat to the reactor or the column or it may be sent to an off-site utility.

In an embodiment, the stabilizer column 160 may be operated at an overhead pressure of about 34 kPa (g) (5 psig) to about 207 kPa (g) (30 psig) and a bottoms temperature of about 65° C. (150° F.) to about 122° C. (250° F.). In an aspect, the heat to the stabilizer column 160 may be supplied from a reboiler 161 using steam.

Referring back to the fractionation column 140, the fractionator bottoms stream in line 143 may be split to provide a first liquid stream in line 144 and a second liquid stream in line 145. The fractionator bottoms stream in line 143 may comprise aromatic hydrocarbons, preferably the fractionator bottoms stream in line 143 may comprise toluene. The first liquid stream in line 144 may be further processed to separate the toluene. In an embodiment, the first liquid stream in line 144 may be passed to the rerun column 170 to separate heavies from the toluene. In an embodiment, the rerun column 170 may be operated at an overhead pressure of below atmospheric pressure or vacuum pressure. In an exemplary embodiment, the rerun column 170 may be operated at an overhead pressure of about −6 kPa (g) (−1 psig) to about −69 kPa (g) (−10 psig) or about 32 kPa (a) (4.7 psia) to about 95 kPa (a) (13.8 psia). In another exemplary embodiment, the rerun column 170 may be operated at atmospheric pressure. In an embodiment, the rerun column 170 may be operated at a bottoms temperature of about 65° C. (150° F.) to about 149° C. (300° F.). In an aspect, the heat to the rerun column 170 may be supplied from a reboiler 171 using steam. A heavy stream is separated in the rerun column 170 bottoms and taken in line 174. In an embodiment, the heavy stream in line 174 may comprise C8-16 aromatics and multi-ring aromatics but may also include some toluene.

A rerun overhead stream comprising toluene is taken in line 172 from the rerun column 170. The rerun overhead stream comprising toluene in line 172 may be cooled in a heat exchanger 173 and a cooled rerun overhead stream is passed to a rerun overhead receiver 175. The rerun overhead stream 172 is further cooled in the rerun overhead receiver 175 to provide a toluene stream in line 176. A vacuum pump 178 is provided at the overhead of the rerun column 170 on the stream 177 from the overhead receiver 175 to operate the rerun column 170 at a pressure as low as possible. The recovered toluene stream in line 176 may be combined with the second liquid stream in line 145 to provide a toluene product stream in line 149.

While the foregoing embodiment envisages the use of methylcyclohexane (MCH) and toluene as the liquid organic hydrogen carrier (LOHC), the alternative, similar LOHC systems may benefit from the disclosed process. Examples may include cyclohexane (CH) and benzene; dimethylcyclohexanes (DMCH) and xylenes; and trimethylcyclohexanes (TMCH) and trimethylbenzenes (TMB).

The foregoing process and apparatus provide an efficient way of producing hydrogen from a hydrocarbonaceous stream.

### Example 1

Boehmite Al₂O₃ base was extruded into a 1/16″ cylinder and calcined to gamma Al2O3 at 500° C. The gamma Al₂O₃ had a surface area of 220 m²/g. This Al₂O₃ base was impregnated with Pt and K, dried, oxidized at 537° C. and then reduced in flowing H₂ at 565° C. This catalyst will be referred to as the dehydrogenation reference catalyst.

### Example 2

Boehmite Al₂O₃ base extruded into a 1/16″ cylinder and calcined to gamma Al₂O₃ at 730° C. The gamma Al₂O₃ had a surface area of 140 m²/g. This Al₂O₃ base was impregnated with Pt and K, dried, oxidized at 537° C. and then reduced in flowing H₂ at 565° C. in the identical manner as the dehydrogenation reference catalyst. This catalyst will be referred to as the dehydrogenation catalyst of the current invention.

### Example 3

A small-scale flow reactor was arranged to perform dehydrogenation. A feed of 99.8% purity methylcyclohexane (MCH) was procured. Conditions were set up in the reactor of 425 C, 4 LHSV, 4H2:HC and 80 psig. MCH conversion to toluene was in excess of 95% for both the dehydrogenation reference catalyst and the current invention dehydrogenation catalyst. Both catalysts produced some C14 heavy material due to toluene dimerization. The dehydrogenation reference catalyst produced 130 ppm of C14 heavy materials. The current invention dehydrogenation catalyst produced 30 ppm of C14 heavy materials. This demonstrates the value of this invention to reduce the amount of heavy formation.

### SPECIFIC EMBODIMENTS

While the following is described in conjunction with specific embodiments, it will be understood that this description is intended to illustrate and not limit the scope of the preceding description and the appended claims.

A first embodiment of the invention is a process of producing aromatic hydrocarbons, comprising contacting a hydrocarbonaceous feed stream with a dehydrogenation catalyst wherein the catalyst has a surface area of less than about 140 m²/g to produce a dehydrogenated effluent stream; and separating the dehydrogenated effluent stream into a vapor stream comprising hydrogen and a liquid stream. An embodiment of the invention is one, any or all of prior embodiments in this paragraph up through the first embodiment in this paragraph wherein the catalyst has a selectivity to the dehydrogenation of methylcyclohexane to toluene and hydrogen of greater than 99.0%. An embodiment of the invention is one, any or all of prior embodiments in this paragraph up through the first embodiment in this paragraph wherein the catalyst has a surface area from about 90-140 m²/g. An embodiment of the invention is one, any or all of prior embodiments in this paragraph up through the first embodiment in this paragraph wherein the catalyst has a surface area from about 120-140 m²/g. An embodiment of the invention is one, any or all of prior embodiments in this paragraph up through the first embodiment in this paragraph wherein the catalyst comprises a gamma alumina support. An embodiment of the invention is one, any or all of prior embodiments in this paragraph up through the first embodiment in this paragraph wherein the gamma alumina is calcined at a temperature of about 650° C. to 815° C. An embodiment of the invention is one, any or all of prior embodiments in this paragraph up through the first embodiment in this paragraph wherein the catalyst comprises 0.1-2 wt % platinum. An embodiment of the invention is one, any or all of prior embodiments in this paragraph up through the first embodiment in this paragraph wherein the catalyst comprises 0.2-4 wt % alkaline earth or alkaline metal. An embodiment of the invention is one, any or all of prior embodiments in this paragraph up through the first embodiment in this paragraph wherein the catalyst comprises potassium. An embodiment of the invention is one, any or all of prior embodiments in this paragraph up through the first embodiment in this paragraph wherein the catalyst comprises lithium. An embodiment of the invention is one, any or all of prior embodiments in this paragraph up through the first embodiment in this paragraph wherein the catalyst comprises lithium and potassium. An embodiment of the invention is one, any or all of prior embodiments in this paragraph up through the first embodiment in this paragraph wherein the catalyst is oxidized and reduced before being used in the dehydrogenation reactor. An embodiment of the invention is one, any or all of prior embodiments in this paragraph up through the first embodiment in this paragraph, wherein the hydrocarbonaceous feed stream comprises at least one system selected from methylcyclohexane and toluene, cyclohexane and benzene, dimethylcyclohexanes and xylenes or trimethylcyclohexanes and trimethylbenzenes. An embodiment of the invention is one, any or all of prior embodiments in this paragraph up through the first embodiment in this paragraph wherein the hydrocarbonaceous feed stream comprises methylcyclohexane. An embodiment of the invention is one, any or all of prior embodiments in this paragraph up through the first embodiment in this paragraph, wherein the dehydrogenated effluent stream comprises aromatic hydrocarbons. An embodiment of the invention is one, any or all of prior embodiments in this paragraph up through the first embodiment in this paragraph wherein the dehydrogenated effluent stream comprises a concentration of hydrocarbons greater than C10 that it less than about 150 wt ppm higher than the concentration of hydrocarbons greater than C10 in the hydrocarbonaceous feed stream. An embodiment of the invention is one, any or all of prior embodiments in this paragraph up through the first embodiment in this paragraph wherein the dehydrogenated effluent stream comprises a concentration of hydrocarbons greater in size than C10 that it less than about 100 wt ppm higher than the concentration of hydrocarbons greater than C10 in the hydrocarbonaceous feed stream. An embodiment of the invention is one, any or all of prior embodiments in this paragraph up through the first embodiment in this paragraph wherein the dehydrogenated hydrocabonaceous effluent stream comprises a concentration of hydrocarbons greater than C10 that it less than about 50 wt ppm hydrocarbon higher than the concentration of hydrocarbons greater than C10 in the hydrocarbonaceous feed stream.

A second embodiment of the invention is a process of producing aromatic hydrocarbons, comprising contacting a hydrocarbonaceous feed stream comprising at least one system selected from methylcyclohexane and toluene, cyclohexane and benzene, dimethylcyclohexanes and xylenes or trimethylcyclohexanes and trimethylbenzenes with a dehydrogenation catalyst wherein the catalyst has a surface area of less than about 140 m²/g to produce a dehydrogenated effluent stream; and separating the dehydrogenated effluent stream into a vapor stream comprising hydrogen and a liquid stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of producing aromatic hydrocarbons, comprising:
contacting a hydrocarbonaceous feed stream with a dehydrogenation catalyst wherein said catalyst has a surface area of less than about 140 m2/g to produce a dehydrogenated effluent stream; and
separating said dehydrogenated effluent stream into a vapor stream comprising hydrogen and a liquid stream.

2. The process of claim 1 wherein said catalyst has a selectivity to the dehydrogenation of methylcyclohexane to toluene and hydrogen of greater than 99.0%.

3. The process of claim 1 wherein said catalyst has a surface area from about 90-140 m²/g.

4. The process of claim 1 wherein said catalyst has a surface area from about 120-140 m²/g.

5. The process of claim 1 wherein the catalyst comprises a gamma alumina support.

6. The process of claim 5 wherein the gamma alumina is calcined at a temperature of about 650° C. to 815° C.

7. The process of claim 1 wherein the catalyst comprises 0.1-2 wt % platinum.

8. The process of claim 1 wherein the catalyst comprises 0.2-4 wt % alkaline earth or alkaline metal.

9. The process of claim 1 wherein the catalyst comprises potassium.

10. The process of claim 1 wherein the catalyst comprises lithium.

11. The process of claim 6 wherein the catalyst comprises lithium and potassium.

12. The process of claim 1 wherein the catalyst is oxidized and reduced before being used in the dehydrogenation reactor.

13. The process of claim 1, wherein said hydrocarbonaceous feed stream comprises at least one system selected from methylcyclohexane and toluene, cyclohexane and benzene, dimethylcyclohexanes and xylenes or trimethylcyclohexanes and trimethylbenzenes.

14. The process of claim 10 wherein said hydrocarbonaceous feed stream comprises methylcyclohexane.

15. The process of claim 1, wherein said dehydrogenated effluent stream comprises aromatic hydrocarbons.

16. The process of claim 1 wherein said dehydrogenated effluent stream comprises a concentration of hydrocarbons greater than C10 that it less than about 150 wt ppm higher than the concentration of hydrocarbons greater than C10 in the hydrocarbonaceous feed stream.

17. The process of claim 1 wherein said dehydrogenated effluent stream comprises a concentration of hydrocarbons greater in size than C10 that it less than about 100 wt ppm higher than the concentration of hydrocarbons greater than C10 in the hydrocarbonaceous feed stream.

18. The process of claim 1 wherein said dehydrogenated hydrocabonaceous effluent stream comprises a concentration of hydrocarbons greater than C10 that it less than about 50 wt ppm hydrocarbon higher than the concentration of hydrocarbons greater than C10 in the hydrocarbonaceous feed stream.

19. A process of producing aromatic hydrocarbons, comprising:
contacting a hydrocarbonaceous feed stream comprising at least one system selected from methylcyclohexane and toluene, cyclohexane and benzene, dimethylcyclohexanes and xylenes or trimethylcyclohexanes and trimethylbenzenes with a dehydrogenation catalyst wherein said catalyst has a surface area of less than about 140 m2/g to produce a dehydrogenated effluent stream; and
separating said dehydrogenated effluent stream into a vapor stream comprising hydrogen and a liquid stream.
